(19) United States
(12) Patent Application Publication
(10) Pub. No.: US 2018/0021027 A1
(43) Pub. Date: Jan. 25, 2018

(54) BIOPSY BRUSH

(71) Applicant: Boston Scientific Scimed, Inc., Maple Grove, MN (US)

(72) Inventors: Sean P. FLEURY, Minneapolis, MN (US); Paul SMITH, Smithfield, RI (US)

(73) Assignee: Boston Scientific Scimed, Inc., Maple Grove, MN (US)

(21) Appl. No.: 15/651,179

(22) Filed: Jul. 17, 2017

Related U.S. Application Data
(60) Provisional application No. 62/364,932, filed on Jul. 21, 2016.

(51) Int. Cl. A61B 10/02; A61B 10/04

(52) U.S. Cl. CPC A61B 10/02; A61B 10/04; A61B 2010/045; A61B 2010/0216; A61B 2017/320012

(57) ABSTRACT

A medical device configured to collect tissue from within a subject's body includes an elongate shaft having a proximal end, a distal end, and a distal region proximate the distal end. A plurality of bristles may be positioned on the distal region to form a brush. The distal region may be configured to deform upon activation to increase a cross-sectional dimension of the brush. The cross-sectional dimension of the brush may be the largest dimension of the brush in a direction transverse to a longitudinal axis of the shaft.

### CROSS-REFERENCE TO RELATED APPLICATIONS

This patent application claims the benefit of priority under 35 U.S.C. §119 to U.S. Provisional Patent Application No. 62/364,932, filed Jul. 21, 2016, which is herein incorporated by reference in its entirety.

### TECHNICAL FIELD

The present disclosure relates generally to medical devices. More particularly, the disclosure relates to medical devices used, for example, for tissue collection during biopsy, and methods for using the devices.

### BACKGROUND

Biopsy involves the extraction of tissue (or cells) from within the body of a patient for analysis to determine the presence or extent of a disease. One method of extracting tissue from within the patient's body is endoscopic biopsy. During endoscopic biopsy, one or more medical devices (e.g., needles, forceps, brushes, etc.), inserted into the body through the lumen of an endoscope (or a catheter or other like device), is used to acquire and extract tissue from within the body. While a tissue collection device in the form of a brush may be the most benign method of tissue removal during some biopsies, its inability to collect a sufficient amount of tissue for analysis may discourage its use. The systems and methods described herein may alleviate this deficiency. The scope of the current disclosure, however, is defined by the attached claims, and not by the ability to solve any specific problem.

### SUMMARY

Aspects of the present disclosure relate to, among other things, a tissue collection device for biopsy applications. These aspects may include one or more of the features described below.

In one aspect of the present disclosure, a medical device configured to collect tissue from within a body of a subject is disclosed. The medical device may include an elongate shaft having a proximal end, a distal end, and a distal region proximate the distal end. A plurality of bristles may be positioned on the distal region of the shaft to form a brush. The distal region of the shaft may be configured to deform upon activation to increase a cross-sectional dimension of the brush. The cross-sectional dimension of the brush may be a largest dimension of the brush in a direction transverse to a longitudinal axis of the shaft.

Embodiments of the disclosed medical device may include one or more of the features described below. The device may include a sheath having a lumen configured to slidably receive the shaft therein, wherein the cross-sectional dimension of the brush is configured to increase when the brush is extended out of the lumen. In the device, the distal region of the shaft may include at least one articulating joint, wherein portions of the shaft adjacent to the at least one articulating joint may be configured to undergo relative displacement upon the activation. The at least one articulating joint may include a hinge or a notch. The distal region of the shaft may include a plurality of links separated by articulating joints. The distal region of the shaft may be configured to deform upon a first activation to increase the cross-sectional dimension of the brush to a first value and further deform upon a second activation to increase the cross-sectional dimension to a second value greater than the first value. The distal region of the shaft may be configured to transform from a linear configuration prior to the activation to a bent configuration after the activation. The brush may be configured to decrease in length and increase in surface area after activation. The distal region of the shaft may be configured to transform from a linear configuration prior to the activation to one of a substantially U-shaped configuration, a substantially triangular configuration, a substantially L-shaped configuration, and a substantially V-shaped configuration after the activation.

Embodiments of the disclosed medical device may also include one or more of the features described below. The deformation of the shaft from an undeformed configuration to a deformed configuration may increase the cross-sectional dimension of the brush from a first value prior to the activation to a second value after the activation, wherein the distal region is further configured to transform from the deformed configuration to the undeformed configuration to decrease the cross-sectional dimension of the brush from the second value to the first value. The distal region of the shaft may be configured to bend upon the activation to increase the cross-sectional dimension of the brush. The distal region of the shaft may be configured to inflate upon the activation to increase the cross-sectional dimension of the brush. The distal region of the shaft may be configured to be activated by applying tension on one or more activating wires that extend along the shaft from the proximal end to the distal end. The distal region of the shaft may be configured to be activated by applying heat on the distal region. The brush may have a substantially cylindrical shape prior to the activation. The distal end of the shaft may be rounded.

In another aspect of the present disclosure, a method of collecting tissue from within a body of a subject is disclosed. The method may include inserting a medical device into the body. The medical device may include an elongate shaft having a proximal end, a distal end, and a distal region proximate the distal end. A plurality of bristles may be positioned on the distal region of the shaft to form a brush having a cross-sectional dimension of a first value. The cross-sectional dimension of the brush may be a largest dimension of the brush in a direction transverse to a longitudinal axis of the shaft. The method may also include activating the medical device to deform the distal region of shaft such that the cross-sectional dimension of the brush increases from the first value after the activation.

Embodiments of the disclosed method may include one or more of the following aspects. The method may also include rubbing the brush against tissue within the body to collect a tissue sample. The method may also include transforming the distal region of the shaft to decrease the cross-sectional dimension of the brush back to the first value. Inserting the medical device into the body may include introducing the medical device into the body through a lumen of a sheath such that the cross-sectional dimension of the brush is less than the first value, and extending the brush out of the lumen to increase the cross-sectional dimension of the brush to the first value. The distal region of the shaft may include at least one articulating joint, wherein deforming the distal region of shaft may include inducing relative displacement on portions of the shaft adjacent to the at least one articulating joint. Inducing relative displacement may include bending the distal region of the shaft at the at least one articulating joint.

In yet another aspect of the present disclosure, a medical device configured to collect tissue from within a body of a subject is disclosed. The medical device may include a sheath having a lumen extending therethrough, and an elongate shaft configured to be introduced into the body through the lumen. The shaft may have a proximal end, a distal end, and a distal region proximate the distal end. A plurality of bristles may be positioned on the distal region of the shaft to form a brush having a cross-sectional dimension. The cross-sectional dimension may be a largest dimension of the brush in a direction transverse to a longitudinal axis of the shaft. The cross-sectional dimension may be a first value when the brush is positioned within the lumen and a second value, greater than the first value, when the brush is positioned outside the lumen. The device may also include at least one articulating joint on the distal region of the shaft. Upon activation, the distal region of the shaft may be configured to bend at the at least one articulating joint to increase the cross-sectional dimension of the brush from the second value before the activation to a third value after the activation.

Embodiments of the disclosed device may include one or more of the following features. The at least one articulating joint may include at least one of a hinge or a notch. The distal region of the shaft may be configured to transform from a linear configuration prior to the activation to one of a substantially U-shaped configuration, a substantially triangular configuration, a substantially L-shaped configuration, and a substantially V-shaped configuration after the activation. The brush may be configured to decrease in length and increase in surface area after activation

It may be understood that both the foregoing summary and the following detailed description are exemplary and explanatory only, neither being restrictive of the inventions claimed below.

### BRIEF DESCRIPTION OF THE DRAWINGS

The accompanying drawings, which are incorporated in and constitute a part of this specification, illustrate exemplary embodiments that, together with the written description, serve to explain the principles of this disclosure.

FIG. 1 is an exemplary medical device of the current disclosure extending into the body through the distal end of an endoscope;

FIGS. 2A-2C illustrate exemplary embodiments of the medical device of FIG. 1;

FIG. 3A is a side view of the distal end of the medical device of FIG. 2A in an expanded configuration;

FIG. 3B is a side view of the distal end of an exemplary disclosed medical device in its expanded configuration;

FIG. 3C is a side view of the distal end of another exemplary disclosed medical device in its expanded configuration;

FIG. 4A illustrates part of an exemplary distal region of a disclosed medical device;

FIG. 4B illustrates part of another exemplary distal region of a disclosed medical device;

FIG. 4C illustrates part of another exemplary distal region of a disclosed medical device;

FIG. 5 illustrates the distal end of an exemplary disclosed medical device; and

FIG. 6 is a flow chart that illustrates an exemplary method of using a disclosed medical device.

### DETAILED DESCRIPTION

The present disclosure is now described with reference to an exemplary tissue collection device used in an endoscopic biopsy procedure. However, it should be noted that reference to this particular device and procedure is provided only for convenience and not intended to limit the disclosure. A person of ordinary skill in the art would recognize that the concepts underlying the disclosed exemplary device and application method may be utilized in any device or procedure, medical or otherwise. The discussion below uses the terms “proximal” and “distal” to refer to the relative positions of the device and its components. For instance, proximal end 14 refers to a location closer to a user using the device, and distal end refers to a position further away from the user. Further, as used herein, the terms “about,” “approximately” and “substantially” indicate a range of values within +/−10% of a stated value.

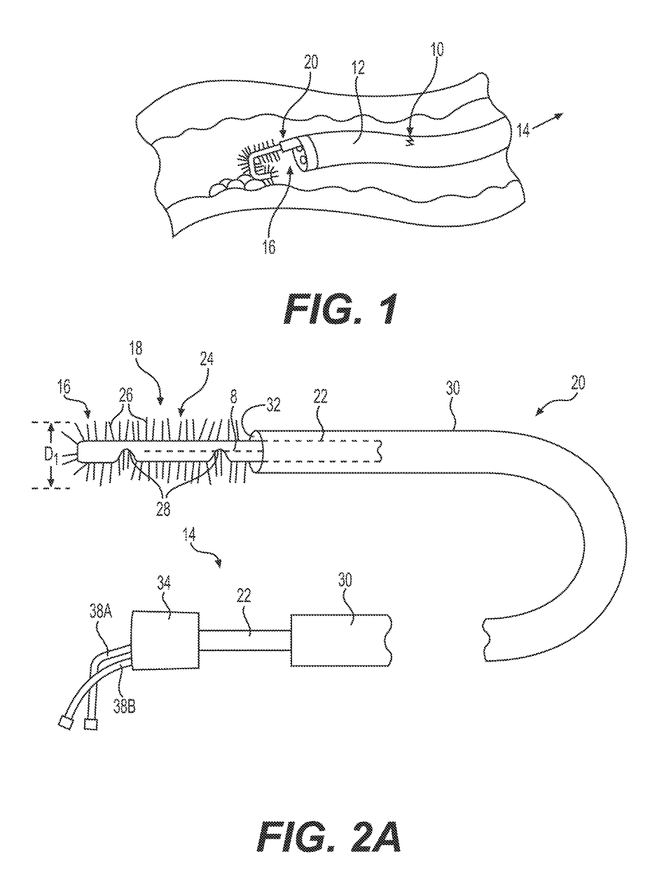

FIG. 1 illustrates an embodiment of the disclosed tissue collection device (device 20) positioned within the body of a patient. In FIG. 1, the device 20 is illustrated as extending into the patient's body (“body”) through the distal end 16 of an endoscope 10. Endoscope 10 may include an elongate flexible tubular section 12 extending from a proximal end 14 positioned outside the body to a distal end 16 positioned proximate a worksite (e.g., the biopsy site) within the body. The device 20 may be inserted into the body from the proximal end 14 through a lumen of the endoscope 10.

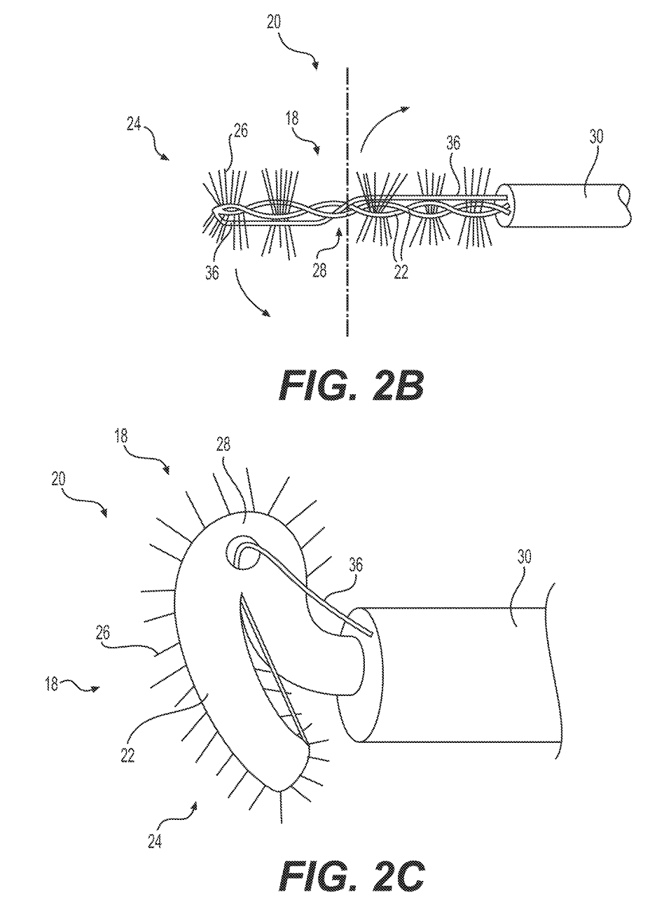

FIG. 2A is an illustration of an exemplary tissue collection device 20 of FIG. 1. Device 20 may be used in combination with a flexible catheter or sheath 30 that extends from the proximal end 14 to the distal end 16. A lumen 32 may extend through the length of the sheath 30 from the proximal end 14 to the distal end 16. In use, the sheath 30 may extend into the patient's body through the lumen of the endoscope 10. Device 20 may include a flexible shaft 22 that slidably extends into the patient's body through the lumen 32 of the sheath 30. Shaft 22 has a longitudinal axis 8 that extends from its proximal end to its distal end. As the device 20 is inserted into the body, the shaft 22 may flex to navigate through tortuous curves in the body cavity. Since a catheter (such as sheath 30) suitable for use with device 20 is well known to people of ordinary skilled in the art, it is not described extensively herein.

Although device 20 is described as being inserted into the body through the lumen of a sheath 30, which in turn is inserted into the body through the lumen of an endoscope 10, this is not a requirement. That is, in some embodiments, the device 20 may be used without an endoscope 10 and/or a sheath 30. For instance, in some embodiments, the distal end 16 of the device 20 may be directly inserted into the body through a body orifice (mouth, nose, etc.), and pushed in until its distal end 16 is suitably positioned proximate the worksite in the body and its proximal end 14 is positioned outside the body. In some embodiments, the endoscope 10 may be eliminated but a sheath 30 may be used. That is, the sheath 30 may be directly inserted into the body and the device 20 may be inserted through the sheath 30. It is also contemplated that, in some embodiments, a sheath 30, with a device 20 positioned therein, may be directly inserted into the body. In some embodiments, device 20 may include one or more radiographic markers (not shown) which are discernable relative to living tissue when viewed under a fluoroscope. These radiographic markers may assist in proper positioning of the device 20 within the body.

In some embodiments, the proximal end of shaft 22 may include a handle 34 which is positioned outside the body when the device 20 is in use. The handle 34 may be used by a user (technician, etc.) to insert, maneuver, and operate the device 20 in the body. A plurality of brushing elements, or bristles 26, may be disposed in a distal region 18 of the shaft 22, proximate the distal end 16, to form a brush 24. In some embodiments, to insert the device 20 into the body, the device 20 may first be inserted into the lumen 32 of sheath 30. The distal end of the sheath 30, with the device 20 (including brush 24) positioned in its lumen 32, may then be inserted into the body. When the brush 24 is positioned within lumen 32, the bristles 26 of the brush 24 may be pushed and folded towards the longitudinal axis 8 of the shaft 22 by the surrounding walls of lumen 32. This folded configuration of the brush 24 within the lumen 32 is referred to as its retracted configuration. After the sheath 30 is suitably positioned proximate the worksite within the body, the user may push the shaft 22 of device 20 distally (e.g., using handle 34) from the proximal end 14 to push the brush 24 out of the lumen 32. When the brush 24 exits the lumen 32, the bristles 26, now relieved of the constraining force of the lumen walls, spring out, or otherwise transform, to their extended configuration. The length of the bristles 26 may be such that, in its extended configuration, an outer cross-sectional dimension D₁ of the brush 24 transverse to the longitudinal axis 8 is greater than the corresponding dimension of the brush 24 in its retracted configuration (or the diameter of lumen 32). After capturing tissue from the worksite, the user may pull the shaft 22 proximally to retract the brush 24 into the lumen 32 (i.e., transition the brush 24 to its retracted configuration), prior to withdrawal of the device 20 from the body.

The shaft 22 may be made of any material (nitinol, stainless steel, polymer, nylon, etc.) and may have any size and cross-sectional shape (e.g., substantially circular cross-section, rectangular, triangular, square, polygonal, elliptical, oblong, etc.). In general, the size of the shaft 22 (e.g., diameter, length, etc.) may depend on the patient's anatomy, and/or the type of procedure being performed. In some embodiments, the shaft 22 may include one or more wires or strands twisted together (see, for example, shaft 22 of FIG. 2B). In some embodiments, the shaft 22 may have a configuration similar to a flexible hollow tube. In general, the shaft 22 may have sufficient flexibility to allow it to bend during insertion (and withdrawal) of the device 20 into the patient's body. The shaft 22 may also have sufficient rigidity to prevent kinking when it is pulled and pushed (to extend and retract the brush 24).

The distal region 18 of the shaft 22 (where the brush 24 is disposed) may extend along a length of the shaft 22 at its distal end 16. Although FIG. 2A illustrates the brush 24 as extending to the distal-most end of shaft 22, this is not a requirement. In some embodiments, the distal-most end of shaft 22 may include a rounded (or atraumatic) tip member, and the brush 24 may be positioned proximal to the tip member. The bristles 26 may be formed on shaft 22 in any known manner. In some embodiments, one end of each bristle 26 may be attached to the distal region 18 to form brush 24. In some embodiments, one end of each bristle 26 may be anchored (e.g., held in an interference fit in a cavity) to the distal region 18. In embodiments where the shaft 22 is formed of twisted wire, the bristles 26 may be clamped between the twisted wires. It is also contemplated that, in some embodiments, the bristles 26 may be formed integrally with (i.e., as one single part with) the shaft 22. In general, the bristles 26 may have any cross-sectional shape (e.g., substantially circular cross-section, rectangular, triangular, square, polygonal, elliptical, oblong, etc.).

After the brush 24 is extended into the worksite, the bristles 26 may be used to rub against the tissue at the worksite to separate and capture cells from the tissue between the bristles 26. While the particular dimensions (length, diameter, etc.) and structural properties (stiffness, etc.) of the bristles 26 depend on the application, in general, these dimensions and properties may be suitable to acquire tissue by scraping. The bristles 26 may be made of any bio-compatible and flexible material known in the art (e.g., nitinol, stainless steel, nylon, polymer, and/or any suitable material or combination of materials). In some embodiments, all the bristles 26 of brush 24 may be made of the same material, and these bristles 26 may have substantially the same dimensions (length, diameter, etc.). In some embodiments, brush 24 may have a substantially cylindrical cross-sectional shape in its extended configuration (configuration of FIG. 2A). However, other shapes (conical, irregular, etc.) of the basket 24 are also contemplated.

In some embodiments, different regions of the brush 24 may have a different stiffness. That is, brush 24 may include groups of bristles 26 made of different materials and/or having different dimensions. In some embodiments, the bristles 26 may be made of filaments having suitable properties. The filaments may be monofilaments (a filament made of a single material) formed by extrusion or multicomponent filaments (a core about which one or more layers of material are concentrically arranged) formed by co-extrusion. In some embodiments, some or all exposed surfaces of at least some of the bristles 26 may be patterned (textured, etc.) to improve tissue acquisition and/or retention capability.

The bristles 26 may be arranged substantially completely around the distal region 18 of the shaft 22, or may be arranged on selected surfaces (e.g., one side) of the distal region 18. The bristles 26 of brush 24 may radiate radially outwards from the shaft 22. In general, the bristles 26 may radiate at an angle (i.e., right angle, acute angle, obtuse angle) relative to the longitudinal axis 8 of shaft 22. In some embodiments, substantially all the bristles 26 may radiate from the shaft 22 at substantially the same angle (see FIG. 1), while in other embodiments, the bristles 26 may radiate outward from the shaft 22 at different angles (see FIG. 2A). It is also contemplated that, in some embodiments, the bristles 26 in different portions of the brush 24 may radiate outward at different angles.

As explained previously, when brush 24 exits the lumen 32 of sheath 30, it transforms from its retracted configuration to its extended configuration. As the brush 24 transforms to its extended configuration, the bristles 26 rotate away from the shaft 22 (i.e., the angle which the bristles 26 make with longitudinal axis 8 increases), and a cross-sectional dimension of the brush 24 transverse to longitudinal axis 8 increases. This increased cross-sectional dimension of the brush 24 in its extended configuration (as compared to its retracted configuration) increases the cross-sectional area of the brush 24, and that surface area of the brush 24 that is exposed to (or available for contact with) tissue.

In this disclosure, the term cross-sectional dimension is used to refer to the outer dimension (or size) of the brush 24 in a direction transverse to the longitudinal axis 8 of the shaft 22 at a location where shaft 22 enters the sheath 30. That is, the cross-sectional dimension of the brush 24 is the largest dimension of the brush 24 in a direction transverse to longitudinal axis 8. For example, if the brush 24 has a cylindrical configuration in its extended configuration, its cross-sectional dimension in the extended configuration is the outer diameter of the brush 24. When the brush 24 is viewed from the distal end 16 towards the proximal end 14, an increased cross-sectional dimension increases the viewed area of the brush 24 (or the cross-section area of the brush 24 transverse to the viewing direction). In other words, an increased cross-sectional dimension increases the distally facing area of the brush 24. When the brush 24 is used to scrape tissue at a worksite in a tubular body cavity (e.g., in the passageways of the lungs), an increased cross-sectional dimension increases the contact area and/or contact pressure of the bristles 26 with the tubular tissue walls. That is, an increased cross-sectional dimension of the brush 24 increases the contact area, or the surface area, of the brush with tissue. The increase in contact area and/or contact pressure (resulting from an increased cross-sectional dimension) increases the amount of tissue that can be collected by the brush 24.

The capability of the brush 24 to acquire tissue (e.g., a greater amount of tissue) increases with increasing cross-sectional dimension. However, a bigger brush 24 (e.g., one having a greater cross-sectional dimension) may be difficult to introduce into, and extract from, a tortuous body cavity. Therefore, in some embodiments, the brush 24 may be configured to selectively transform from its extended configuration to an expanded configuration when it is desired to further increase its cross-sectional dimension (distally facing area, cross-sectional area, surface area configured to contact tissue, etc.). That is, the cross-sectional dimension of the brush 24 (and therefore, its distally facing area, cross-sectional area, surface area configured to contact tissue, etc.) in the expanded configuration may be greater than its corresponding cross-sectional dimension D₁ in the extended configuration.

In some embodiments, the selective transformation of the brush 24 may be enabled by allowing the shaft 22 that forms the distal region 18 to deform (e.g., fold, bend, buckle, curl, inflate, etc.) upon activation by the user to increase the cross-sectional dimension of the brush 24. In some embodiments, one or more articulating joints 28 may be provided in the distal region 18 of the shaft 22 to enable deformation of the distal region 18. In some embodiments, upon activation by the user, portions of the shaft 22 on either side of each articulating joint 28 may undergo relative displacement (e.g., fold or bend) to transform the brush 24 to its expanded configuration and consequently increase its cross-sectional dimension. Although articulating joint 28 of FIG. 2A is illustrated as a geometric discontinuity (i.e., a notch) on shaft 22, this is merely exemplary. In general, an articulating joint 28 can be any region (i.e., even a region without a geometric discontinuity) of shaft 22 that deforms upon activation by the user such that portions of the shaft 22 on either side of the articulating joint undergo relative displacement.

FIG. 2B illustrates another embodiment of a medical device 20 of the present disclosure. Similar to device 20 of FIG. 2A, the distal region 18 of a shaft 22 that extends out of the sheath 30 includes a brush 24 with bristles 26. The shaft 22 of FIG. 2B is made of a plurality of wires (two wires in the illustrated embodiment) twisted or intertwined together. The bristles 26 may be attached to the twisted wires. As illustrated in FIG. 2B, the wires may be twisted together with a gap therebetween. An activating wire 36, attached to the distal end 16 of the shaft 22, may extend to the proximal end 14 through the sheath 30. The shaft 22 of the multifilament brush 24 may buckle to transform the brush 24 to its expanded configuration when an activating wire that runs alongside the shaft 22 is pulled from the proximal end 14. In the distal region 18, the activating wire 36 may pass from one side of the shaft 22 to the opposite side through a space between the twisted wires of the shaft 22. When the activating wire 36 is pulled from the proximal end 14, the force on the wire 36 cause the shaft 22 at the distal region 18 to buckle and bend. The bending of the shaft 22 increases the surface area of the brush 24, thus transforming the brush 24 to its expanded configuration. Upon activation by the user, the shaft 22 may bend at a region (i.e., articulating joint 28) where the activating wire 36 passes (through the shaft 22) from one side of the shaft 22 to the opposite side.

FIG. 2C illustrates another embodiment of a device 20 having a shaft 22 that buckles to transform a multifilament brush 24 to an expanded configuration when an activating wire 36 that runs alongside the shaft 22 is pulled. In the embodiment of FIG. 2C, the shaft 22 may be a single element (wire, rod, shaft, etc.) that extends out of the sheath 30. The brush 24 may be formed by bristles 26 attached to the distal region 18 of the shaft 22. The shaft 22 may include a hole in the distal region 18. The activating wire 36, attached to the distal end 16 of the shaft 22 may pass from one side of the shaft 22 to the opposite side through the hole, and extend proximally through the sheath 30. When the user pulls the activating wire 36 at the proximal end 14, the distal portion 18 of the shaft 22 may buckle at an articulating joint 28 (formed in the region of the hole) to transform the brush 24 to its expanded configuration.

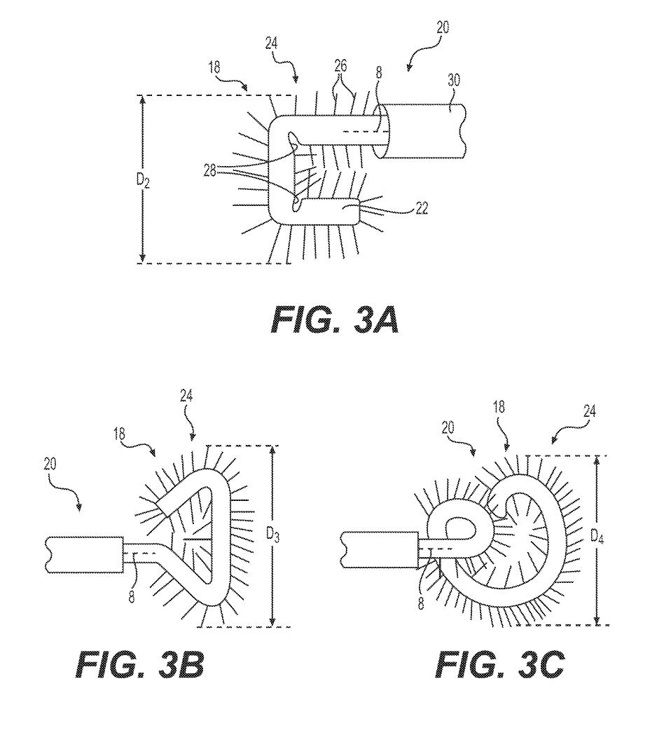

FIG. 3A illustrates an exemplary brush 20 transformed to an expanded configuration from its extended configuration (illustrated in FIG. 2A). In the expanded configuration, the brush 24 has a cross-sectional dimension D₂ which is greater than its cross-sectional dimension D₁ in the extended configuration. In some embodiments, the shaft 22 may bend at the articulating joints 28 upon activation by the user from the proximal end 14. The articulating joints 28 may be activated jointly or separately. For example, in some embodiments, upon activation by the user, all the articulating joints 28 of shaft 22 may bend in a predetermined manner to transform the brush 24 to its expanded configuration. In the exemplary brush 20 illustrated in FIG. 3A, upon activation, the shaft 22 bends by about 90° at its two articulating joints 28 to transform the distal region 18 of the shaft 22 into a substantially U-shaped configuration, and thus increase the cross-sectional dimension of the brush 24 from D₁ (in FIG. 2A) to D₂ (as illustrated in FIGS. 2A and 3A, D₂≅2D₁). In some embodiments, some or all of the articulating joint 28 may be activated separately by the user. For example, upon first activation, some of the articulating joints 28 may bend to transform the brush 24 to a first expanded configuration, and upon a second activation, the remaining articulating joints 28 may bend to transform the brush 24 from the first expanded configuration to a second expanded configuration.

Upon activation, the distal region 18 of the shaft 22 may transform to any configuration or shape to transform the brush 24 to an expanded configuration and thus increase its outer cross-sectional dimension. Although a substantially U-shaped configuration is illustrated in FIG. 3A, this is only exemplary. In general, the distal region 18 may transform from a substantially straight configuration prior to the activation to any bent configuration (e.g., substantially U-shaped configuration, a substantially triangular configuration, a substantially L-shaped configuration, substantially C-shaped, substantially e-shaped, substantially V-shaped configuration, etc.) after the activation. FIGS. 3B and 3C illustrate other exemplary embodiments of a device 20 where the distal region 18 of the shaft 22 is configured to transform into a different shape. In the embodiment of FIG. 3B, upon activation, the articulating joints 28 on shaft 22 bend by different amounts to transform the distal region 18 to a substantially triangular configuration. As the distal region 18 deforms from a substantially straight configuration (see FIG. 2A) to a substantially triangular configuration (see FIG. 3B), the brush 24 transforms from an extended configuration to an expanded configuration. The cross-sectional dimension of the brush 24 in this expanded configuration is D₃, which is greater than its corresponding cross-sectional dimension in the extended configuration prior to the transformation (i.e., D₁, see FIG. 2A). In the embodiment of the device 20 illustrated in FIG. 3C, upon activation by the user, the distal region 18 of the shaft 22 deforms to a coil-shaped configuration. When the distal region 18 deforms to the coil-shaped configuration, the brush 24 transforms to its expanded configuration with an increased cross-sectional dimension D₄ as compared to D₁.

The device 20 may be activated in any manner to transform the brush 24 from the extended configuration to the expanded configuration. In some embodiments, the device 20 may be activated using activating wires (activating wires 38A, 38B of FIG. 2A and activating wire 36 of FIGS. 2B and 2C) that extend along the length of the shaft 22 to its proximal end 14. The activating wires may be made of flexible wires, cables, or rods that may be configured to transmit a force (pulling, pushing, etc.) from the proximal end 14 of the shaft 22 to its distal end 16. The shaft 22 may be configured to bend at the articulating joints 28 when a pulling or a pushing force is applied to the activating wires 38A, 38B. In some embodiments, when a user of the device 20 wants to transform the brush 24 to its expanded configuration, the user may pull on one or both the activating wires 38A, 38B to bend the distal region 18 of the shaft 22 at the articulating joints 28. For example, in some embodiments, the shaft 22 may be a hollow tube, and the activating wires 38A, 38B may be pull wires that extend longitudinally along the inner surface of the tubular shaft 22. These activating wires 38A, 38B may be attached to the shaft 22, in the distal region 18, distal to an articulating joint 28. In use, when an activating wire 38A, 38B is pulled, the activating wire applies a pulling force to the distal region 18 at the attachment point and causes the shaft 22 to bend at the articulating joint 28. Since the use of activating wires (e.g., pull wires) to bend or articulate distal ends of medical devices are well known in the art, they will not be described in more detail herein.

Alternatively or additionally, in some embodiments, the shaft 22 may be activated by other methods. For example, in some embodiments, the shaft 22 may be made of a shape memory alloy having a stress-free state in which the distal region 18 is straight. Pulling on the activating wires 38A, 38B may force the distal region 18 to transform to a bend configuration thus transforming the brush 24 to an expanded configuration. Releasing the activating wires 38A, 38B may allow the distal region 18 to return to its stress-free configuration and the brush 24 back to its extended configuration. It is also contemplated that, in some embodiments, thermal and/or electrical energy may be used to bend the shaft 22 at the articulating joints 28. For example, in some embodiments, the articulating joints 28 may have a structure similar to a bi-material thermostat. To activate such an articulating joint 28, electric current may be used to heat the articulating joint 28 (e.g., by joule heating). As a result of differential thermal expansion of the different materials of the articulating joint 28, it may bend and transform the brush 24 to its expanded configuration. Stopping the current flow may cause the articulating joints 28 to transform to their original configuration and the brush 24 back to its extended configuration.

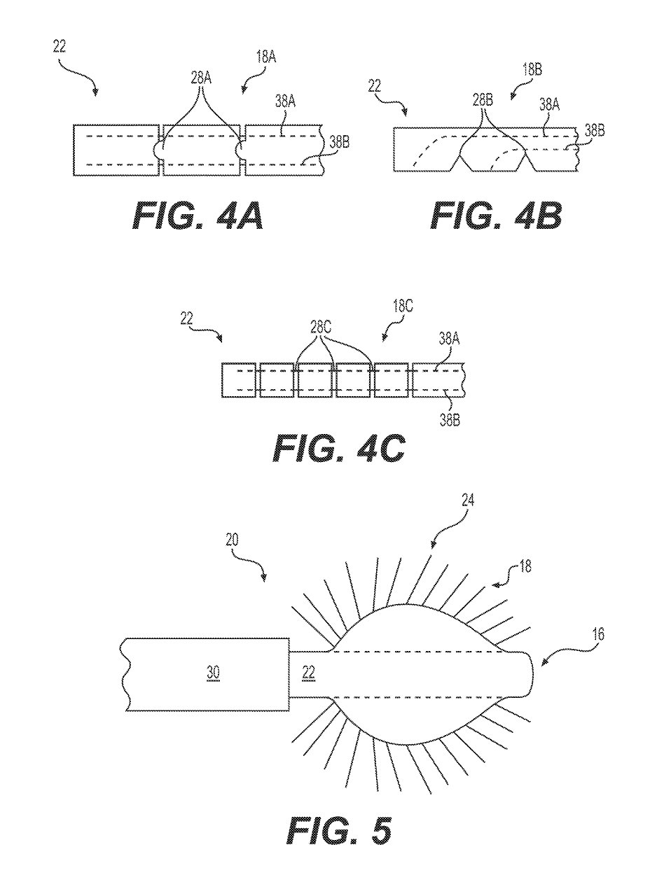

An articulating joint 28 may be any region of the shaft (or any feature) that enables portions of the shaft 22 on either side of the region/feature to bend, or deflect, relative to each other, upon activation by the user. FIGS. 4A-4C illustrate some exemplary embodiments of articulating joints that may be used in the distal region 18 of the shaft 22. The bristles 26 normally disposed in the distal region 18 are not shown in FIGS. 4A-4C for clarity. In the embodiment of FIG. 4A, the distal region 18A is formed by multiple sections that are connected together by articulating joints 28A in the form of hinges or pivots. Activating wires 38A and 38B, connected to these shaft sections, may extend to the proximal end of the device 20. The user may pull one or both of these activating wires 38A, 38B to bend the distal region 18A (e.g., cause portions of the shaft adjacent to the pivots to deflect relative to each other), and transform the brush 24 to its expanded configuration. In some embodiments, pulling one of the activating wires (e.g., 38A) may transform the distal region 18A to a first bend configuration that corresponds to a first expanded configuration of the brush 24, and pulling the second activating wire (e.g., 38B) may then transform the distal region 18A to a second bend configuration that corresponds to a second expanded configuration of the brush 24. In some embodiments, the distal region 18A may be biased to remain straight. When the activating wires 38A, 38B are pulled, the distal region 18A may bend at the pivots against the biasing force. Upon release of the activating wires 38A, 38B, the distal region 18A may transform back to its original straight configuration.

As illustrated in FIG. 4B, in some embodiments, notches (or living hinges) may be provided in the distal region 18B of the shaft 22 to form articulating joints 28B. The distal region 18B may be configured to bend at these notches in response to the pulling of the activating wires 38A, 38B from the proximal end. The dimensions of the notches may be sufficient to bend the distal region 18B as desired without causing rupture of shaft 22. In some embodiments, the portion of the distal region 18B at the notches may be made of a material which is more flexible (e.g., rubber) than other portions of the distal region 18B, to selectively bend the shaft 22 at the notches when the activating wires 38A, 38B are pulled. In some embodiments, pulling one activating wire 38A may bend the distal region 18B in one direction to transform the brush 24 to its expanded configuration. And, pulling the other activating wire 38B may bend the distal region 18B in the opposite direction to restore the brush 24 to its extended configuration. In some embodiments, as described with reference to FIG. 4A, the distal region 18B may be biased to remain straight, and may return to its straight configuration when the force on the activating wires 38A, 38B is released.

FIG. 4C illustrates an embodiment where the distal region 18C of the shaft 22 is formed by multiple links (solid or hollow tubes) connected together by activating wires 38A, 38B to form articulating joints 28C between each of the links. Pulling the activating wires 38A, 38B may cause each of the links to move with respect to its adjacent links to transform the brush 24 to its expanded configuration. Any of the techniques described with reference to FIGS. 4A and 4B may be used to bend and restore the distal region 18C.

In the discussion above, the distal region 18 of the shaft 22 is described as bending at one or more discrete articulating joints 28 to transform the brush 24 to its expanded configuration. However, this is not a requirement. In some embodiments, upon activation by the user, a length of the distal region 18 may change to a different configuration (for example, see FIG. 3C) to transform the brush 24 to its expanded configuration.

FIG. 5 illustrates an embodiment of a device 20 in which the distal region 18 of the shaft 22 is inflatable. In some such embodiments, the distal region 18 may be formed of a material (e.g., more compliant, inflatable, etc.) different from other regions of the shaft 22. After extending the brush 24 out of the sheath 30 to transform it to its extended configuration, the user may transform the brush 24 to its expanded configuration by activating the flow of a fluid (air, liquid, etc.) to the distal end 16 of the device 20. The fluid may inflate the inflatable distal region 18 and thus transform the brush 24 to its expanded configuration. After a sufficient amount of tissue has been collected from the worksite using the brush 24 in its expanded configuration, the user may transform the brush 24 back to its extended configuration by discharging the fluid from the shaft 22.

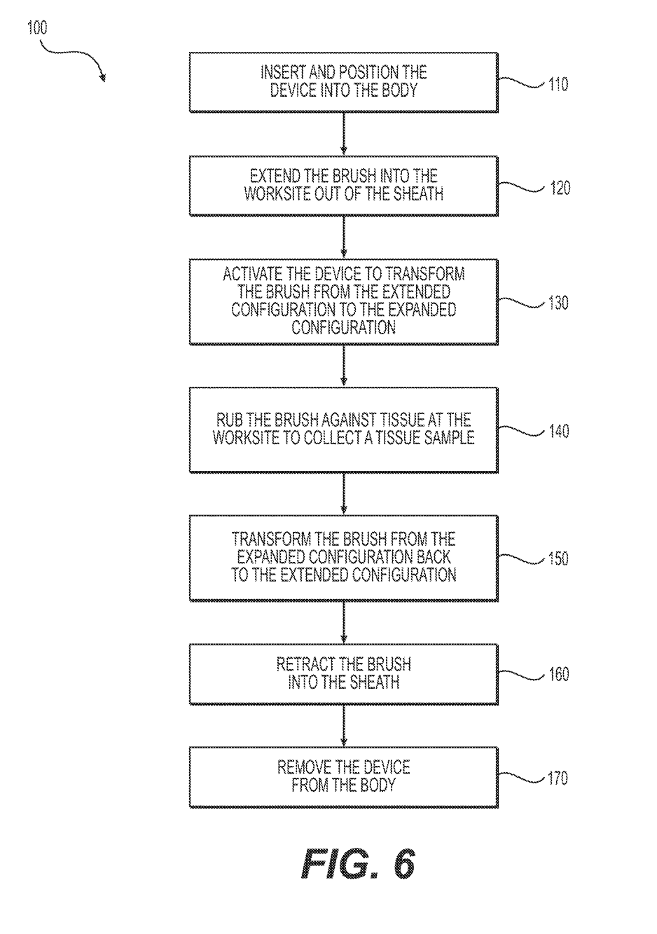

FIG. 6 is a flow chart illustrating an exemplary method 100 of using device 20. In the discussion that follows, a method of using the device 20 to acquire a tissue sample from a worksite within the body of a patient is described. However, it should be noted that this method is only exemplary, and the device 20 may be used for any suitable procedure. The device 20 is first inserted into the body and suitably positioned (step 110). The distal end 16 of the device 20 may be inserted into a body cavity through an orifice and pushed in until the distal end 16 is positioned proximate the worksite from where the tissue sample is to be acquired. When the device 20 is inserted into the body, the brush 24 of the device 20 is positioned within the sheath 30 in its retracted configuration. After the device 20 is suitably positioned, the brush 24 is extended out of the sheath 30 (step 120). In some embodiments, the shaft 22 of the device 20 may be pushed distally from the proximal end 14 to extend the brush 24 out of the sheath 30. As the brush 24 extends out of the sheath 30, it transforms from the retracted configuration within the sheath 30 to an extended configuration outside the sheath 30.

In the extended configuration, the outer cross-sectional dimension of the brush 24 is greater than its corresponding cross-sectional dimension in the retracted configuration. The device 20 may then be activated to transform the brush 24 from the extended configuration to the expanded configuration (step 130). The user may activate the device 20 by pulling on (or tensioning) the activating wires 38A, 38B that extend through the device 20. Upon activation, the distal region 18 of the shaft 22, upon which the brush 24 is disposed, deforms (e.g., folds, bends, expands, inflates, etc.) to transform the brush 24 to the expanded configuration. In the expanded configuration, a cross-sectional dimension of the brush 24 at its greatest extent is greater than its corresponding cross-sectional dimension in the extended configuration.

The brush 24 is then rubbed against the tissue at the worksite to collect a tissue sample (tissue cells, etc.) on, or between, the bristles 26 of the brush 24 (step 140). The brush 24 is then transformed to its extended configuration from the expanded configuration (step 150). In some embodiments, the brush 24 may be transformed back to its extended configuration by releasing the tension of the activating wires 38A, 38B. The brush 24 with the tissue sample is retracted into the sheath 30 (step 160), and the device 30 is removed from the body.

Using the endoscopic biopsy method described above, a sufficient volume of target tissue may be separated and removed from the body. In some embodiments, the volume of the tissue removed may be controlled by controlling the outermost cross-sectional dimension of the brush 24 in the expanded configuration. Performing the biopsy procedure using a brush simplifies the procedure and reduces the possibility of medical complications.

Other examples of the present disclosure will be apparent to those skilled in the art from consideration of the specification and practice of the present disclosure disclosed herein. It is intended that the specification and examples be considered as exemplary only, with a true scope and spirit of the present disclosure being indicated by the following claims.

We claim:

1. A medical device configured to collect tissue from within a body of a subject, comprising:
an elongate shaft having a proximal end, a distal end, and a distal region proximate the distal end; and
a plurality of bristles positioned on the distal region of the shaft to form a brush, wherein the distal region of the shaft is configured to deform upon activation to increase a cross-sectional dimension of the brush, wherein the cross-sectional dimension of the brush is a largest dimension of the brush in a direction transverse to a longitudinal axis of the shaft.

2. The device of claim 1, further including a sheath having a lumen configured to slidably receive the shaft therein, wherein the cross-sectional dimension of the brush is configured to increase when the brush is extended out of the lumen.

3. The device of claim 1, wherein the distal region of the shaft includes at least one articulating joint, wherein portions of the shaft adjacent to the at least one articulating joint are configured to undergo relative displacement upon the activation.

4. The device of claim 1, wherein the at least one articulating joint includes a hinge or a notch.

5. The device of claim 1, comprising a plurality of articulating joints, wherein the distal region of the shaft includes a plurality of links separated from each other by the plurality of articulating joints.

6. The device of claim 1, wherein the distal region of the shaft is configured to deform upon a first activation to increase the cross-sectional dimension of the brush to a first value and further deform upon a second activation to increase the cross-sectional dimension to a second value greater than the first value.

7. The device of claim 1, wherein the distal region of the shaft is configured to transform from a linear configuration prior to the activation to a bent configuration after the activation.

8. The device of claim 1, wherein the brush is configured to decrease in length and increase in surface area after the activation.

9. The device of claim 1, wherein deformation of the shaft from an undeformed configuration to a deformed configuration increases the cross-sectional dimension of the brush from a first value prior to the activation to a second value after the activation, and wherein the distal region is further configured to transform from the deformed configuration to the undeformed configuration to decrease the cross-sectional dimension of the brush from the second value to the first value.

10. The device of claim 1, wherein the distal region of the shaft is configured to bend upon the activation to increase the cross-sectional dimension of the brush.

11. The device of claim 1, wherein the distal region of the shaft is configured to inflate upon the activation to increase the cross-sectional dimension of the brush.

12. The device of any claim 1, wherein the distal region of the shaft is configured to be activated by applying tension on one or more activating wires that extend along the shaft from the proximal end to the distal end.

13. A method of collecting tissue from within a body of a subject, comprising:
inserting a medical device into the body, the medical device including:
an elongate shaft having a proximal end, a distal end, and a distal region proximate the distal end; and
a plurality of bristles positioned on the distal region of the shaft to form a brush having a cross-sectional dimension of a first value, wherein the cross-sectional dimension of the brush is a largest dimension of the brush in a direction transverse to a longitudinal axis of the shaft;
activating the medical device to deform the distal region of the shaft such that the cross-sectional dimension of the brush increases from the first value after the activation.

14. The method of claim 13, further including:
rubbing the brush against tissue within the body to collect a tissue sample; and
transforming the distal region of the shaft to decrease the cross-sectional dimension of the brush back to the first value.

15. The method of claim 13, wherein inserting the medical device into the body includes:
introducing the medical device into the body through a lumen of a sheath such that the cross-sectional dimension of the brush is less than the first value; and
extending the brush out of the lumen to increase the cross-sectional dimension of the brush to the first value.

16. The method of claim 13, wherein the distal region of the shaft includes at least one articulating joint, and wherein deforming the distal region of shaft includes inducing relative displacement on portions of the shaft adjacent to the at least one articulating joint.

17. The method of claim 16, wherein inducing relative displacement includes bending the distal region of the shaft at the at least one articulating joint.

18. A medical device configured to collect tissue from within a body of a subject, comprising:
a sheath having a lumen extending therethrough;
an elongate shaft configured to be introduced into the body through the lumen, the shaft having a proximal end, a distal end, and a distal region proximate the distal end;
a plurality of bristles positioned on the distal region of the shaft to form a brush having a cross-sectional dimension, wherein the cross-sectional dimension is a largest dimension of the brush in a direction transverse to a longitudinal axis of the shaft, and wherein the cross-sectional dimension is a first value when the brush is positioned within the lumen and a second value, greater than the first value, when the brush is positioned outside the lumen; and
at least one articulating joint on the distal region of the shaft, wherein upon activation, the distal region of the shaft is configured to bend at the at least one articulating joint to increase the cross-sectional dimension of the brush from the second value before the activation to a third value after the activation.

19. The device of claim 18, wherein the at least one articulating joint includes at least one of a hinge or a notch.

20. The device of claim 18, wherein the brush is configured to decrease in length and increase in surface area available to contact with tissue after activation.
